(19) United States
(12) Patent Application Publication
(10) Pub. No.: US 2017/0181619 A1
(43) Pub. Date: Jun. 29, 2017

(54) Vision Testing System and Method For Testing The Eyes

(71) Applicant: Oculus Optikgeraete GmbH, Wetzlar (DE)

(72) Inventors: Andreas Steinmueller, Wettenberg (DE); Stephan Degle, Jena (DE)

(21) Appl. No.: 15/387,079

(22) Filed: Dec. 21, 2016

(30) Foreign Application Priority Data
Dec. 23, 2015 (DE) 10 2015 226 725.3

(51) Int. Cl. A61B 3/032; A61B 3/14; A61B 3/06; A61B 3/04; A61B 3/113; A61B 3/11; A61B 3/00; A61B 3/028

(52) U.S. Cl. CPC A61B 3/032; A61B 3/0008; A61B 3/0075; A61B 3/14; A61B 3/0285; A61B 3/04; A61B 3/113; A61B 3/111; A61B 3/112; A61B 3/066

(57) ABSTRACT

The invention relates to a vision testing system (10) and to a method for testing the eyes of a subject, comprising a display device (11, 12, 13) by means of which optotypes can be visualized to at least one eye of the subject, comprising a control device (14) for controlling the display device, the display device comprising a backlit screen (19, 30, 35), the screen having an adjusting device for adjusting a screen luminance of the screen to an ambient luminance, the adjusting device having a measuring means for measuring the screen luminance.

The invention relates to a vision testing system and to a method for testing the eyes of a subject, comprising a display device by means of which optotypes can be visualized to at least one eye of the subject, comprising a control device for controlling the display device, the display device comprising a backlit screen, the screen having an adjusting device for adjusting a screen luminance of the screen to an ambient luminance.

Vision testing systems of this kind are sufficiently known and are routinely used to perform eyesight tests. While refraction values of a subject can be objectively determined using an aberrometer, the refraction values subjectively perceived as optimal by the subject might differ from the objectively determined refraction values. For example, a divergence of the objective and refractive refraction values is observed in situations where the subjective refraction values are determined under mesopic or scotopic lighting conditions. In particular, the refraction values objectively determined under mesopic vision or scotopic vision can deviate from refraction values determined subjectively under the same lighting conditions.

To establish these lighting conditions during an eyesight test, optotypes are presented to the subject by means of a backlit screen and at the same time a screen luminance of the screen and an ambient luminance are lowered, among other measures. Accordingly, the room in which the eyesight test is carried out is darkened and at the same time a screen background of the optotypes is displayed more darkly as well so as to be able to simulate the desired visual conditions during mesopic vision or scotopic vision. Furthermore, the known display devices or screens additionally have a control device via which an operator can control a rendering of optotypes on the screen. The control device can be realized in the manner of a remote control, for example. Depending on the type of screen, said screen can have linear or also a circular polarization. The polarization of the screen is routinely used to carry out eyesight tests in connection with a trial frame or a phoropter. The camera device can be used to measure the eyes, for example, measuring being barely possible in the case of simulated mesopic vision or scotopic vision due to the lighting conditions.

Screens that can adjust their screen luminance or background luminance as a function of an ambient luminance are already known. For instance, some mobile phones are equipped with this function. In the case of the known screens, an optoelectronic sensor measures light incident on the sensor or an ambient luminance, and a screen luminance of the screen is controlled or adjusted correspondingly via the background lighting of the screen. In case of increasing ambient luminance, for example, a screen luminance is increased as well, and vice-versa. Adjusting devices of this kind have the disadvantage, however, that the discrete electronic components of the adjusting device, such as the optoelectronic sensor, typically have a nonlinear characteristic in terms of their response behavior. Thus, the screen luminance to an ambient luminance is not adjusted proportionally, but according to a function that is determined by the functions of the respective electronic components of the adjusting device. This nonlinear function of the adjustment of the screen luminance to the ambient luminance thus distorts a result of the subjective refraction determination under changing, different lighting conditions.

Therefore, the object of the present invention is to provide a vision testing system and a method for testing the eyes of a subject with a vision testing system by means of which more precise results can be achieved in eyesight test.

This object is attained by a vision testing system having the features of claim 1 and by a method having the features of claim 11.

The vision testing system according to the invention for testing the eyes of a subject comprises a display device by means of which optotypes can be visualized to at least one eye of the subject, and a control device for controlling the display device, the display device comprising a backlit screen, the screen having an adjusting device for adjusting a screen luminance of the screen to an ambient luminance, the adjusting device having a measuring means for measuring the screen luminance.

Controlling a background lighting of the screen and thus the screen luminance becomes possible in particular owing to the fact that the adjusting device has the measuring means by means of which the screen luminance of the screen can be measured. The measuring means allows constant checking as to whether the measured screen luminance corresponds to a screen luminance required at an ambient luminance. If the measured screen luminance deviates from the required screen luminance, the screen luminance can be corrected correspondingly by means of the adjusting device so that the measured screen luminance corresponds to the required screen luminance. Consequently, the screen luminance can be adjusted proportionally or according to a linear characteristic to an ambient luminance by means of the adjusting device independent of potential characteristics of discrete electronic components of the display device, allowing eyesight tests to be carried out even more precisely owing to the consistent display conditions of optotypes under varying ambient luminance. In principle, the screen can also be configured for linear or circular polarization, for example, or to have another means usable for image separation.

In one embodiment, the measuring means can have an optoelectronic sensor, which can be arranged adjacent to or in front of a display surface of the screen in such a manner that the screen luminance can be measured. For example, the optoelectronic sensor can be arranged in a longitudinal side or corner of a frame of the screen in such a manner that light emitted by the screen is incident on the optoelectronic sensor. The optoelectronic sensor can be arranged in such a manner that it is not in contact with the display surface of the screen immediately in front of the display surface, but that it is merely adjacent to the display surface. Yet, it may also be envisaged that the optoelectronic sensor is arranged immediately in front of the display surface, at a distance from the display surface or in immediate contact with the display surface. This arrangement of the optoelectronic sensor allows measuring a screen luminance of the screen in a comparatively precise manner without the optoelectronic sensor being visible in a visually disturbing manner in front of the display surface.

The measuring means can have another optoelectronic sensor by means of which the ambient luminance can be measured. The other optoelectronic sensor can also be arranged in a frame of the screen or at another point of the display device. The substantial aspect is that light emitted by the screen is not incident on the other optoelectronic sensor because this might distort a measuring result. Altogether, it thus becomes possible to measure the ambient luminance and the screen luminance and to control them accordingly.

For a display device, the vision testing system can have a stationary distance-test display device, whose display-surface size is configured for eyesight tests at a viewing distance of 3 m to 10 m, preferably of 4 m to 8 m, and/or a mobile near-test display device, whose display-surface size is configured for eyesight tests at a viewing distance of 10 cm to 3 m, preferably of 30 cm to 1 m. The distance-test display device can be used to display optotypes for testing distance vision and the near-test display device can be used to display optotypes for testing near vision. The vision testing system can have either the distance-test display device or the near-test display device and other display devices, if applicable, or it can have both the distance-test display device and the near-test display device and other display devices, if applicable. The distance-test display device can preferably be set up stationarily in the above-indicated viewing distance relative to the subject or can be mounted on a wall. If the subject is placed in a defined viewing distance relative to the distance-test display device in order to preform eyesight tests, the viewing distance to the distance-test display device can be precisely determined. Also, in this case, a display-surface size of the distance-test display device can be many times larger than a display-surface size of the neartest display device because comparatively larger optotypes may be presented on the display surface of the distance-test display device. Owing to the mobile nature of the near-test display device, it can be held or placed by an operator or by the subject at almost any distance within the above-indicated viewing distance relative to the eyes of the subject. Corresponding eyesight tests can thus be carried out at different viewing distances from the near-test display device. Both the distance-test display device and the near-test display device can be remotely controlled by an operator by means of the control device.

The control device can be a mobile phone or a tablet computer. These control devices are equipped with a touch-sensitive screen, via which an operator can comfortably select different eyesight tests or optotypes and assign them to the display device for presentation. Furthermore, it is possible for the control device to be programmed in such a manner that control of the display device is performed entirely by the control device; i.e., in this case, the display device merely serves to present the optotypes initiated by the control device. Independently of the display device, it is also possible in this case to input changes regarding the optotypes or new eyesight tests into the control device through a software update of the control device, while this is not required by the display device. The control device can communicate wirelessly with the display device via Wi-Fi or Bluetooth. However, the control device can also be a stationary computer or a laptop on which software for controlling the display device can be executed.

The display device can have a camera device by means of which the eyes of the subject can be captured. The camera device can be a digital camera or a camera chip having a lens and being integrated in a frame of the screen. When the eyes of the subject are captured, a pupillary distance, a lighting-dependent pupil diameter, a measuring distance, a head tilt and/or a line of sight or a fixation of the eyes of the subject can be registered and measured by means of the camera device. This information can be used further in the course of eyesight tests.

The display device can have an illuminating device having an infrared light source by means of which the eyes of the subject can be illuminated. In particular if the eyesight tests are performed under mesopic or scotopic light conditions, reduced ambient brightness makes it difficult to capture the eyes of a subject with a camera device in order to perform certain eyesight tests. With the infrared light source, the eyes of the subject can be illuminated with infrared light independently of an ambient illumination and can be captured by means of a correspondingly adapted camera device. Advantageously, dazzling of the subject by the illumination of the eyes with infrared light can also be avoided in this way. It may also be envisaged that the illuminating device comprises multiple infrared light sources, such as IR light-emitting diodes. The infrared light sources can be arranged immediately adjacent to a camera device. Preferably, in this case, two infrared light sources can be arranged next to the camera device equidistantly in relation thereto in one plane with the eyes of the subject. It is also possible to arrange infrared light sources in the frame of the display device. Overall, it thus becomes possible to find out whether the refraction values determined objectively under mesopic vision or scotopic vision deviate from the refraction values determined subjectively under the same lighting conditions and thus at the same pupil diameters.

For instance, the camera device and/or the infrared light source can also be moved into a storage position in the display device or into a capturing position outside of the display device. Furthermore, the camera device and/or the infrared light source can be arranged at the screen in such a manner that the camera can be folded away into a storage position, such as behind the screen, or can be brought into a capturing position next to the screen for camera recording. The camera can be moved from the storage position into the capturing position and back by a drive unit of the camera device. Furthermore, a deflecting prism can be provided if a relatively large camera is used, allowing the camera to be arranged behind the screen in a space-saving manner.

Furthermore, the display device can have a dazzling device, by means of which the eyes of the subject can be illuminated. The dazzling device can comprise at least one light source, which is arranged adjacent to the screen. The light source can be a light-emitting diode, for example. The light source can be integrated in a frame of the display device. It may furthermore be envisaged for one light source of the dazzling device to be arranged on each longitudinal side of the screen of the display device.

The vision testing system can comprise a phoropter or a trial frame. In this case, it becomes possible to determine a refraction of each of the eyes of a subject. The phoropter or the trial frame can have color filters or polarization filters, each of which is adjusted to a color rendering and/or to a polarization of the screen, allowing monocular and binocular eyesight tests to be performed. If a phoropter or a trial frame having linear or circular polarization is already available, for example, the display device of the vision testing system can be selected such that its polarization matches the phoropter or the trial frame. Thus, there is no need to correct a polarization with a λ/4 film, for example.

Moreover, the vision testing system can comprise a building appliance for light control which can be controlled by the control device. For example, the building appliance can be an electrically operated blind, a shutter and/or artificial lighting of an interior room in which the vision testing system is used. In this case, a shutter can be opened or closed and interior lighting can be power-controlled through the control device, for example. The control device can control the building appliance via Wi-Fi or Bluetooth, for example, allowing an operator to comfortably control lighting conditions in the examination room by means of the control device during operation of the display device to perform certain eyesight tests. Among other things, it may also be envisaged that when an operator selects a certain eyesight test at the control device, the building appliances are automatically controlled by the control device to adapt the lighting conditions to the eyesight test.

In the method according to the invention for testing the eyes of a subject with a vision testing system, optotypes visualized to at least one eye of the subject are displayed by a display device of the vision testing system, the display device being controlled by means of a control device of the vision testing system, the display device comprising a backlit screen, a screen luminance of the screen being adjusted to an ambient luminance, the screen luminance being adjusted proportionally as a function of the ambient luminance.

In the method according to the invention, the screen luminance is consequently adjusted to the ambient luminance by means of an adjusting device of the screen in such a manner that a relationship between the screen luminance and the ambient luminance is always linear. In principle, the screen can also be designed to have linear or circular polarization or to have another means that can be used for image separation, for example. As for the advantages of the method according to the invention, reference is made to the description of advantages of the vision testing system according to the invention.

In one embodiment of the method, the screen luminance and the color rendering of a display surface of the screen can be measured by means of an optoelectronic sensor and can be controlled by means of an adjusting device of the screen. In this way, eyesight tests that require a defined presentation of a color or of a contrast of optotypes can be performed in a particularly precise manner. The screen luminance and the color rendering can be controlled automatically by means of the adjusting device, the screen thus calibrating itself. For example, the screen can be a screen of a tablet computer or of a conventional television set. Optotypes can be displayed at a background lighting of the screen of 90 to 300 cd/m² screen luminance. While a display of a background of the optotypes in gray shades is possible, it is not necessary since the screen luminance can be easily adjusted by controlling the background lighting.

A pupillary distance, a pupil diameter, a measuring distance, a head tilt and/or a line of sight of eyes of the subject can be registered and measured by means of a camera device. If a viewing distance between the subject and the screen is basically known because placement of the screen and the subject is stationary, the pupillary distance can be calculated by means of image processing from an image of both eyes of the subject captured by the camera device or by a camera. A relative distance of the pupils can be used to adjust glasses or to present certain eyesight tests. The pupil diameter can also be measured as a function of an ambient lighting in such a way. Vice-versa, if a pupillary distance is known, a measuring distance or viewing distance of the subject relative to the screen can be calculated by means of image processing from an image captured by the camera device. Also, a head tilt of the subject relative to the screen and a line of sight or a fixation on optotypes can be registered.

A pupil diameter can be measured by means of a camera device of the display device, allowing an eyesight test to be performed under mesopic vision or scotopic vision of a subject. In this way, it becomes possible only by means of an illumination of the pupils with infrared light by an illumination device to measure the pupil diameter and to determine subjective refraction under mesopic or scotopic visual conditions by means of an eyesight test. Then the subjective refraction values can be compared to refraction values measured objectively at substantially the same pupil diameter. For example, the objective refraction values can be automatically transmitted to the vision testing system from a measuring device for determining objective refraction values.

For instance, it is also particularly advantageous if a position, in particular a tilt of the screen relative to the eyes of the subject can be measured by means of a position sensor of the display device. The position sensor can by a gyroscopic sensor, via which a spatial position or situation of the screen or of the display surface can be determined. If the eyes of the subject or the head of the subject are/is captured with a camera device of the display device, for example, a tilt of the screen relative to the eyes can be easily calculated by taking into account a known pupillary distance. In this case, the fact that the screen is tilted relative to the eyes and an eyesight test cannot be performed, for example, can also be displayed via the screen. Also, information regarding correct alignment of the screen relative to the eyes of the subject can be provided via the screen. In this way, the subject may be capable of putting the screen in the correct position relative to the subject's eyes as required by the eyesight test by himself/herself.

The eyes of the subject can also be continuously tracked by means of a camera device of the display device. Accordingly, a fixation point of the eyes on a display surface of the screen can be calculated. This becomes possible if a line of sight of the eyes of the subject is registered. In this way, the extent to which the subject dynamically tracks optotypes presented monocularly or binocularly can be examined in the course of eyesight tests.

If there is continuous eye tracking for presented optotypes, a monocular and/or binocular vision performance can be determined from an interrelation between eye movement and optotype position. Eye tracking can also be used when texts presented on the screen are being read.

If a viewing distance or a measuring distance is known, the optotypes can be presented in a size adjusted to the measuring distance. The size-adjusted optotypes can be presented automatically and manually via the control device and by an operator, respectively. In this way, the optotypes are always displayed in the required size and mistakes during the performance of eyesight tests are avoided.

For a display device, the vision testing system can have a stationary distance-test display device and a mobile near-test display device, a pupillary distance and/or a pupil diameter measured with the distance-test display device being usable when measuring with the near-test display device. If a subject is placed in front of the distance-test display device at a defined viewing distance or measuring distance, the measuring distance is thus known. A pupillary distance of the subject can then be measured by means of a camera device of the display device. The pupillary distance can be determined from a camera image by image processing as the measuring distance is known. It is also possible to determine a lighting-dependent pupil diameter in this way. The pupillary distance and/or the pupil diameter can be used when measuring with the near-test display device in such a manner that a measuring distance or a viewing distance of the eyes of a subject from the screen of the near-test display device is calculated. If the near-test display device also has a camera device, a pupillary distance and a pupil diameter of the subject can be registered from an image of the camera device by means of image processing and can be put in relation to the pupillary distance and pupil diameter measured with the distance-test display device, allowing the measuring distance to be calculated in relation to the image capture of the near-test display device or its camera device. Calculations of this kind can be performed by means of triangulation, for example, and can be executed by the control device. In this case, optotypes can always be presented as a function of an actual measuring distance of the near-test display device on the screen thereof, for example.

A measuring distance of eyes of a subject relative to the screen of the near-test display device can be determined even more precisely if the pupillary distance measured with the distance-test display device at a known measuring distance is used to measure the distance with a camera device of the near-test display device, wherein a convergence of the eyes can be taken into account. Since at measuring distances of 10 cm to 3 m optotypes may no longer be observed at infinity when using the near-test display device, the eyes of the subject will focus on an optotype that is presented on a screen of the near-test display device. Visual axes of the eyes will be substantially convergent. This will result in a decreased pupillary distance in comparison to a pupillary distance measured during a distance test or when looking at infinity, and said decreased pupillary distance can be taken into account when calculating a measuring distance and/or performing eyesight tests.

By visualizing the optotypes, an eyesight test can be performed, an eye offset, monocular vision, binocular vision, photopic vision, mesopic vision or scotopic vision of a subject being determinable by said eyesight test. The eye offset can be determined with a Maddox or Thorington eyesight test, for example. Regarding monocular and binocular vision, a refraction of the eyes can also be determined in connection with a trial frame or a phoropter. Aside from the performance of eyesight tests at photopic light or lighting conditions, mesopic or scotopic light or lighting conditions can be set, as well, in order to test or determine a subject's mesopic vision and scotopic vision. To do so, the screen luminance and the ambient luminance can in particular be lowered until mesopic or scotopic visual conditions are established. For example, so-called night driving glasses can be adapted to the subject when his/her pupils are dilated in this case.

The optotypes can be visualized in a size that is adjusted to objectively measured refraction values of a subject, and a subjective review of the objectively measured refraction values can take place, wherein a pupil diameter can be taken into account in this review. Since the objectively measured refraction values may have been obtained by means of an aberrometer at uniform light conditions at a small pupil diameter, for example, the screen luminance and the ambient luminance can be reduced far enough during subjective examination for the pupil diameter to become larger in comparison. This may lead to subjectively measured refraction values that deviate from the objectively measured refraction values. A direct comparison with objectively and with subjectively measured refraction values becomes possible if the optotypes are visualized in a size that is adapted to the objectively measured refraction values of the subject. For this purpose, it can be envisaged that the objectively measured refraction values are transmitted to the control device or are entered into the same, the control device thus automatically selecting the size of the optotypes to be displayed as a function of the objective refraction values.

By visualizing the optotypes, a phoria of a subject can be determined, the phoria being determinable solely by shifting at least one optotype visible to the right eye alone and by shifting at least one optotype visible to the left eye alone relative to each other. A dissociated phoria can be determined with a Maddox or Thorington eyesight test. For this purpose, illuminants, such as light-emitting diodes, arranged outside of the screen, such as in a frame of the display device, can be used in connection with a scale displayed by the screen. Also, owing to the fact that optotypes may be perceived separately by the right and left eye and may be moved separately, a phoria can be determined with the aid of a trial frame or of a phoropter having a polarization film through relative shifting without having to use prisms.

Advantageously, by visualizing the optotypes, an eyesight test can be performed in which the optotypes can be embedded into an image replication of a real environmental situation of a subject. Said real environmental situation can be a depiction of a landscape from which a perspective view of the landscape arises. The optotypes can be depicted within said landscape.

Furthermore, the environmental situation can be a traffic situation in sunshine, fog, rain, twilight or at night with or without display of artificial lighting. For example, a vehicle on a road can be displayed, and the optotypes can be embedded into a license plate. The image replication can be enlarged or reduced until a larger subjective viewing distance from the displayed environmental situation is established. In this way, it is simple to test whether a subject is still able to read the license plate of the vehicle within a freely selectable distance from the vehicle, for example. This eyesight test can be varied with other lighting conditions as described above.

Lights of a vehicle can be displayed as well, a color vision deficiency being determinable by varying a shade of color of the lights. The lights of the vehicle can be tail lights or brake lights, for example, a shade of color being varied in a mutually independent manner. For example, the shade of color can be a shade of red, allowing a color perception of said shade of color to be bridged by two differently varied lights.

The environmental situation can also be displayed so as to be perceived three-dimensionally. For example, the screen can be formed by a conventional television set that is suitable for three-dimensional display in connection with polarization glasses or a trial frame having a polarization filter, for example. However, it may also be envisaged for the environmental situation to be displayed in two dimensions. It is particularly advantageous if a screen is used that can display the environmental situation and/or the optotypes in 4K format.

Other embodiments of the method become apparent from the dependent claims referring back to device claim 1.

Hereinafter, a preferred embodiment of the invention will be explained in more detail with reference to the accompanying drawings.

In the drawings:

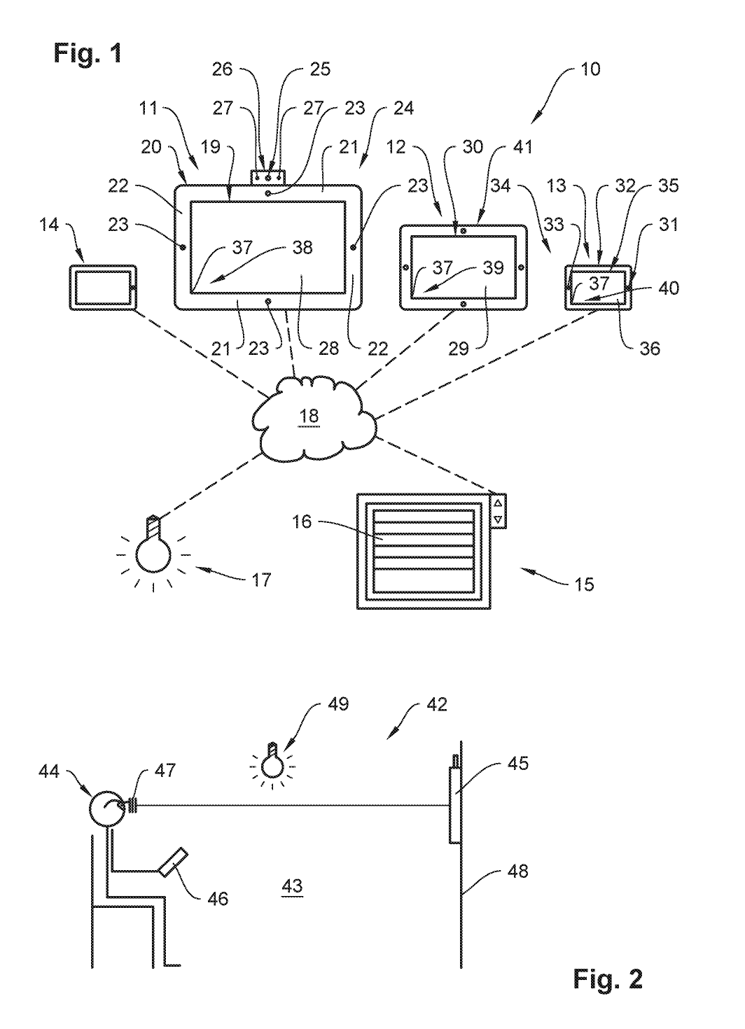

FIG. 1 shows a schematic illustration of an embodiment of a vision testing system; and

FIG. 2 shows a schematic illustration of an arrangement of a vision testing system.

FIG. 1 shows a vision testing system 10 comprising a distance-test display device 11, another distance-test display device 12 and a near-test display device 13 and a control device 14 for controlling the display devices 11, 12 and 13. Furthermore, the vision testing system 10 comprises a shutter control 15 having a shutter 16, and a controllable light source 17 of a room (not illustrated). The shutter control 15 and the light source 17 can also be controlled by means of the control device 14. The display devices 11, 12 and 13 and the shutter control 15 and the light source 17 are connected to the control device 14 via a Wi-Fi network 18 to exchange data. The distance-test display device 11 is formed by a screen 19 having a frame 20. The screen 19 is backlit and can be linearly or circularly polarized. In longitudinal sides 21 and 22 of the frame, light-emitting diodes 23 are integrated, which together form a dazzling device 24. By means of the light-emitting diodes 23, eyes of a subject can be illuminated in such a manner that a dazzling effect is achieved.

The distance-test display device 11 further has a camera device 25 and an illuminating device 26 having infrared light-emitting diodes 27. The camera device 25 can be lowered into the frame 20 together with the illuminating device 26 by motor. A display surface 28 of the screen 11 is larger in comparison to a display surface 29 of a screen 30 of the distance-test display device 12. The distance-test display device 11 can thus be used for larger measuring distances or viewing distances compared to the distance-test display device 12.

The near-test display device 13 is also equipped with a camera device 31 in a frame 32 and with a light-emitting diode 33 forming a dazzling device 34. A display surface 36 of a screen 35 of the near-test display device 13 is small compared to the display surface 29 of the distance-test display device 12. Optotypes or eyesight tests (not illustrated) can be displayed by choice on the respective display surfaces 28, 29 and 36 via the control device 14. Furthermore, the display devices 11, 12 and 13 each have optoelectronic sensors 37, which are arranged in a corner 38, 39 and 40 of the frames 20, 32 and 41. The optoelectronic sensors 37 are part of a measuring means (not illustrated) by means of which a screen luminance of each of the screens 19, 30 and 35 and an ambient luminance of a room are measured. The ambient luminance is measured by means of an optoelectronic sensor (not illustrated) which is integrated in each of the frames 20, 32 and 41. The screen luminance is adjusted to the ambient luminance by means of the control device 14 or by the display device 11, 12 and 13, said adjustment being proportional. The ambient luminance itself can be manually or automatically adjusted by manipulating the shutter 16 and the light source 17 via the control device 14.

FIG. 2 shows a vision testing system 42 in a room 43 together with a subject 44. The vision testing system 42 comprises a distance-test display device 45 and a near-test display device 46 and a trial frame 47, which is worn by the subject 44. The distance-test display device 45 is mounted in a stationary manner on a wall 48 and the near-test display device 46 is manually held by the subject 44. Furthermore, the room 43 is equipped with a light source 49 via which an ambient luminance can be controlled. An operator can perform an eyesight test with the subject 44 via a control device (not illustrated) of the vision testing system 42 by having optotypes presented on the distance-test display device 45, which is located at a comparatively large measuring distance in relation to the subject 44. The near-test display device 46 can be handled by the subject 44 himself/herself, a measuring distance between the subject 44 and the near-test display device 46 being comparatively smaller. In this case, too, optotypes can be presented to the subject 44 as needed via the control device.

1. A vision testing system for testing the eyes of a subject, comprising:
a display device for displaying optotypes to at least one eye of the subject comprising a backlit screen, an adjusting device for adjusting a screen luminance of the screen to an ambient luminance, and a measuring device for measuring the screen luminance; and
a control device for controlling the display device.

2. The vision testing system according to claim 1,
the measuring device comprises an optoelectronic sensor arranged adjacent to or in front of a display surface of the screen in such a manner that the screen luminance can be measured.

3. The vision testing system according to claim 2,
wherein the measuring device comprises a second optoelectronic sensor configured to measure the ambient luminance.

4. The vision testing system according to claim 1,
wherein the display device comprises at least one of a stationary distance-test display device whose display-surface size is configured for eyesight tests at a viewing distance of 3 m to 10 m, and a mobile near-test display device whose display-surface size is configured for eyesight tests at a viewing distance of 10 cm to 3 m.

5. The vision testing system according to claim 1,
wherein the control device comprises at least one of a mobile phone and a tablet computer.

6. The vision testing system according to claim 1,
wherein the display device has a camera device for capturing the eyes of the subject.

7. The vision testing system according to claim 1,
wherein the display device has an illuminating device having an infrared light source configured to illuminate the eyes of the subject.

8. The vision testing system according to claim 1,
wherein the display device further comprises a dazzling device configured to illuminate at least one eye of the subject.

9. The vision testing system according to claim 1,
wherein the vision testing system further comprises at least one of a phoropter and a trial frame.

10. The vision testing system according to claim 1,
wherein the vision testing system further comprises a building appliance for light control configured to be controlled by the control device.

11. A method for testing the eyes of a subject with a vision testing system including a display device comprising a backlit screen having a luminance that is adjustable to an ambient luminance, and a control device controlling the display device the method comprising the steps of:
displaying optotypes on the display; and
adjusting the screen luminance proportionally as a function of the ambient luminance.

12. The method according to claim 11,
wherein the vision testing device further comprises an adjusting device and an optoelectric sensor, and further comprising the steps of adjusting at least one of the screen luminance and a color rendering of a display surface of the screen with the adjusting device, and measuring at least one of the screen luminance and a color rendering of the display surface with the optoelectronic sensor.

13. The method according to claim 11,
further comprising the steps of registering and measuring at least one of a pupillary distance, a pupil diameter, a measuring distance, a head tilt and a line of sight of the eyes of the subject with a camera device of the display device.

14. The method according to claim 11,
further comprising the step of measuring tilt of the screen relative to the eyes of the subject with a position sensor of the display device.

15. The method according to claim 11,
further comprising the step of continuously tracking at least one eye of the subject with a camera device of the display device.

16. The method according to claim 15,
further comprising the step of determining, at least one of a monocular and a binocular visual performance from an interrelation between eye movement and optotype position during the continuous eye tracking.

17. The method according to claim 11,
further comprising the step of adjusting a size of the at least one optotype adjusted corresponding to the measuring distance.

18. The method according to claim 11,
wherein the display device comprises a stationary distance-test display device and a mobile near-test display device, and further comprising the steps of measuring at least one of a pupillary distance and a pupil diameter with the distance-test display device when measuring with the near-test display device.

19. The method according to claim 18,
wherein the camera device corresponds to the near-test display device, and further comprising the step of accounting for a convergence of the eyes while measuring the pupillary distance with the distance-test display device at a known measuring distance with a camera device of the near-test display device.

20. The method according to claim 11,
further comprising the step of performing an eyesight test by visualizing one or more optotypes, and determining at least one of eye offset, monocular vision, binocular vision, photopic vision, mesopic vision and scotopic vision of a subject.

21. The method according to claim 11, further comprising the steps of evaluating a pupil diameter of the subject, and displaying optotypes in a size that is adjusted to measured refraction values of the subject.

22. The method according to claim 11, further comprising the step of determining a phoria of a subject the phoria being determined solely by shifting at least one optotype visible to the right eye alone and by shifting and at least one optotype visible to the left eye alone relative to each other.

23. The method according to claim 11,
further comprising the steps of embedding the at least one optotype in an image replication of a real environmental situation and performing an eyesight test.

24. The method according to claim 23,
wherein the environmental situation is a traffic situation in at least one of sunshine, fog, rain, twilight and night.

25. The method according to claim 23,
wherein the environmental situation is a traffic situation, further comprising the step of determining a color vision deficiency by varying a shade of color of the lights of a vehicle in the environmental situation.

26. The method according to claim 23,
further comprising the step of displaying the environmental situation as three-dimensional.
